(19) United States
(12) Patent Application Publication
(10) Pub. No.: US 2010/0057114 A1
(43) Pub. Date: Mar. 4, 2010

(54) PEEK Spinal Mesh and PEEK Spinal Mesh Applicator

(76) Inventor: Michael S. Butler, St. Charles, IL (US)

Correspondence Address: Bruce J. Bowman;Bowman & Associates, Suite 106, 1016 3rd Ave. SW, Carmel, IN, 46032, US

(21) Appl. No.: 12/552,708

(22) Filed: Sep. 2, 2009

Related U.S. Application Data
(60) Provisional application No. 61/093,644, filed on Sep. 2, 2008.

(51) Int. Cl. A61B 17/00; A61F 2/44

(52) U.S. Cl. 606/151; 623/17.11

(57) ABSTRACT

A bio-compatible covering such as a mesh is used in spinal applications, such as during spinal surgery, to cover, shroud and/or encapsulate at least a portion of one or more vertebrae and/or inter-vertebral devices. The bio-compatible covering preferably, but not necessarily, is formed of PEEK (polyetheretherketone). A delivery instrument for the present PEEK mesh is also provided that places the PEEK mesh onto spinal surfaces (e.g. vertebra and implants) and then applies fasteners to the PEEK mesh and the spinal surfaces for holding the PEEK mesh to the spinal surfaces. The PEEK mesh delivery instrument is preferably, but not necessarily, a minimally invasive delivery instrument (e.g. laproscopic device). The delivery instrument provides a method of simultaneous installation and anchoring of the PEEK mesh. The mesh may be used to emulate (replace) and/or supplement spinal ligaments particularly after spinal surgery such as spinal implant surgery. The mesh may be used in this manner with respect to and/or for the anterior longitudinal ligament and the posterior longitudinal ligament (i.e. artificial ligament). The mesh may also be used as artificial annulus fibrosus material in order to supplement a patient's natural annuls fibrosus. Alternatively or additionally, the mesh may be used to repair or mend a patient's annulus fibrosus.

### RELATED APPLICATIONS

This patent application claims the benefit of and/or priority to U.S. Provisional Patent Application Ser. No. 61/093,644 filed Sep. 2, 2008, entitled “PEEK Spinal Mesh and PEEK Spinal Mesh Applicator” the entire contents of which is specifically incorporated herein by this reference.

### BACKGROUND OF THE INVENTION

1. Field of the Invention

The present invention relates to devices and/or materials for use in spinal surgery including spinal surgery methods.

2. Background Information

During spinal surgery, many anatomical elements are destroyed through direct surgical removal, destruction and/or post-surgical scarring. Upon the destruction of such elements, overall, the spine is mechanically destabilized. It is therefore necessary to re-stabilize the spine. Also, some artificial structures that are implanted into or onto the spine do not have the means to mechanically hold the structure in place while the healing process occurs. Of the structures that do afford some mechanical holding of the structure in place, they are lacking with respect to ease of use, workability and other issues. The lack of mechanical holding and/or inferior mechanical holding can lead to device expulsion and/or failure.

Given the above, it would be desirable to have a manner of mechanically holding a spinal structure and/or spinal implant in place while the healing process occurs.

Given the above, it is also thus desirable to have a manner of mechanically aiding in retaining spinal structures and/or spinal implants in place while the healing process occurs.

### SUMMARY OF THE INVENTION

A bio-compatible covering such as a mesh, net, netting, web, webbing, bag, weave, mat, fiber mat, or the like (collectively, “mesh”) is used in spinal applications, such as during spinal surgery, to cover, shroud and/or encapsulate at least a portion of one or more vertebrae and/or inter-vertebral devices. Preferably, but not necessarily, the present bio-compatible covering comprises a PEEK (polyetheretherketone) mesh, net, netting, web, webbing, bag or the like. A delivery instrument for the present PEEK mesh is also provided that places the PEEK mesh onto spinal surfaces (e.g. vertebra and implants) and then applies fasteners to the PEEK mesh and the spinal surfaces for holding the PEEK mesh to the spinal surfaces. The present PEEK mesh delivery instrument is preferably, but not necessarily, a minimally invasive delivery instrument (e.g. laproscopic device). The delivery instrument provides a method of simultaneous installation and anchoring of the PEEK mesh.

The bio-compatible mesh covering is an artificial material such as a woven material. In addition to PEEK, the mesh may be formed of polyethylene, or any other biocompatible weavable material. A woven polyethylene may be a tightly woven ultrahigh molecular weight polyethylene (UHMWPE) mesh. All meshes provide flexibility and fluid porosity at the surgical site.

The covering is secured to the spinal surfaces/structures (e.g. a vertebra and/or inter-vertebral device) by various means. Such securing methods include, but are not limited to, staples, screws and bio-compatible adhesives, wiring and/or tying means. Other fasteners and/or fastening methods may be used.

In one form, the covering constitutes a bag or the like, of bio-compatible mesh material that encapsulates, covers and/or shrouds at least a portion of a vertebra of a spinal column and/or at least a portion of a spinal implant such as a prosthetic disc. The bag may encapsulate: 1) the whole superior/inferior area of a vertebra; 2) portions of the superior/inferior area of a vertebra; 3) portions of an inter-body or inter-vertebral implant; 4) the entire inter-body/inter-vertebral implant; and/or 5) the whole facet joint capsule or portions thereof.

The mesh may be used to cover an entry way of an implanted prosthetic disc or other inter-body/inter-vertebral device into the spine. When the present mesh is used in conjunction with a spinal implant such as a prosthetic disc, it is optimal to cover the spinal entry way either partially or completely. The present mesh aids in stabilizing the surgical site, adds foundation or structure to the surgical site and/or spinal implant, and/or aids in maintaining at least a portion of a spinal implant in a proper orientation or placement.

In one form, the present mesh may be used to emulate (replace) and/or supplement spinal ligaments particularly after spinal surgery such as spinal implant surgery. The present mesh may be used in this manner with respect to and/or for the anterior longitudinal ligament and the posterior longitudinal ligament (i.e. artificial ligament).

In another form, the present mesh may also be used as artificial annulus fibrosus material in order to supplement a patient's natural annuls fibrosus. Alternatively or additionally, the present mesh may be used to repair or mend a patient's annulus fibrosus.

One purpose of the invention is to work in concert or stand alone as an adjunctive therapy to repair or replace the destroyed structures of the spine and/or as a mechanical retention means to retain an artificial device in the desired implant location. The invention has the ability to be fixated onto or into the spine either over or within the vertebral body, pedicles, facets and/or disc. The device is designed to be adhered to an autonomous and/or artificial spinal structure to provide mechanical support while the healing process takes place.

The mesh can be used for, although is not limited to; repair or replacement of the anterior longitudinal ligament, posterior longitudinal ligament, any segment or all of the disc-annulus fibrosis, as a facet joint capsule or covering, and/or to retain an artificial structure, such as an artificial disc or vertebral body replacement device, within the intervertebral space.

The device or article may also be used to encapsulate and stabilize a bony graft implant that may be used as part of the spinal procedure. The device may also be used as a medicament delivery system while in situ, whereby the mesh structure is either impregnated with a medicament or medicaments, or is mechanically utilized to release medicament(s) from the mesh. Such medicaments, therapeutic agents, include but are not limited to, one or more of the following: culture media, growth factors, differentiation factors, morphogenic proteins, hydrogels, polymers, antibiotics, anti-inflammatory medications, immunosuppressive medications, therapeutically enhanced cells, genetic agents, stem cells, resorbable culture medium, tissue growth or differentiation factors (recombinant generated morphogenetic proteins, PDGF, TGF-.beta., EGF/TGF-.alpha., IGF-I, .beta.FGF), hydrogels, resorbable or nonresorbable synthetic or natural polymers (collagen, fibrin, polyglycolic acid, polylactic acid, polytetrafluoroethylene, etc.). The mesh may also be formed of a bioresorbable material in addition to providing the medicament(s) delivery.

### BRIEF DESCRIPTION OF THE DRAWINGS

The above mentioned and other features and objects of this invention, and the manner of attaining them, will become more apparent and the inventions will be better understood by reference to the following description of embodiments of the invention taken in conjunction with the accompanying drawings, wherein:

FIG. 1 is a side view of a portion of a human spine wherein an artificial or prosthetic spinal disc is disposed between adjacent vertebrae of the spine, other spinal discs between the other vertebrae being natural;

FIG. 2A is an enlarged perspective view of the upper and lower vertebrae adjacent the prosthetic disc of FIG. 1 wherein PEEK spinal mesh has been applied thereto via the present PEEK spinal mesh applicator (see FIG. 22) all in accordance with the principles of the present invention;

FIG. 2B is a top view of the upper vertebrae of FIG. 2A showing a manner in which the present PEEK spinal mesh as applied extends about the upper vertebrae;

FIG. 3A is an enlarged perspective view of the upper and lower vertebrae adjacent the prosthetic disc of FIG. 1 wherein PEEK spinal mesh is provided in accordance with the principles of the present invention;

FIG. 3B is a top view of the upper vertebrae of FIG. 3A showing a manner in which the present PEEK spinal mesh extends about the upper vertebrae;

FIG. 4A is an enlarged perspective view of the upper and lower vertebrae adjacent the prosthetic disc wherein PEEK spinal mesh is provided in accordance with the principles of the present invention;

FIG. 4B is a top view of the upper vertebrae of FIG. 4A showing a manner in which the present PEEK spinal mesh extends about the upper vertebrae;

FIG. 5A is an enlarged perspective view of the upper and lower vertebrae adjacent the prosthetic disc wherein PEEK spinal mesh is provided in accordance with the principles of the present invention;

FIG. 5B is a top view of the upper vertebrae of FIG. 5A showing a manner in which the present PEEK spinal mesh is situated about the upper vertebrae;

FIG. 6A is an enlarged perspective view of the portion of the spine of FIG. 1 having the artificial disc and, more particularly to the upper and lower vertebrae adjacent the prosthetic disc wherein PEEK spinal mesh is provided in accordance with the principles of the present invention;

FIG. 6B is a top view of the portion of the spine of FIG. 6A;

FIG. 7A is an enlarged perspective view of the portion of the spine of FIG. 1 having the artificial disc and, more particularly to the upper and lower vertebrae adjacent the prosthetic disc wherein PEEK spinal mesh is provided in accordance with the principles of the present invention;

FIG. 7B is a top view of the portion of the spine of FIG. 7A;

FIG. 8A is an enlarged perspective view of the portion of the spine of FIG. 1 having the artificial disc and, more particularly to the upper and lower vertebrae adjacent the prosthetic disc wherein PEEK spinal mesh is provided in accordance with the principles of the present invention;

FIG. 8B is a top view of the portion of the spine of FIG. 8A;

FIG. 9A is an enlarged perspective view of the portion of the spine of FIG. 1 having the artificial disc and, more particularly to a view of the upper and lower vertebrae adjacent the prosthetic disc wherein PEEK spinal mesh is provided in accordance with the principles of the present invention;

FIG. 9B is a top view of the portion of the spine of FIG. 9A;

FIG. 10A is an enlarged perspective view of the portion of the spine of FIG. 1 having the artificial disc and, more particularly to the upper and lower vertebrae adjacent the prosthetic disc wherein PEEK spinal mesh is provided in accordance with the principles of the present invention;

FIG. 10B is a top view of the portion of the spine of FIG. 10A;

FIG. 11 is an enlarged side (medial) view of the portion of the spine of FIG. 1 having the artificial disc and, more particularly to the upper and lower vertebrae adjacent the prosthetic disc wherein PEEK spinal mesh is provided in accordance with the principles of the present invention;

FIG. 12 is an enlarged side (medial) view of the portion of the spine of FIG. 1 having the artificial disc and, more particularly to the upper and lower vertebrae adjacent the prosthetic disc wherein PEEK spinal mesh is provided in accordance with the principles of the present invention;

FIG. 13 is a view of a portion of a prosthetic disc and PEEK spinal mesh in accordance with the present principles particularly illustrating a manner of attachment of the PEEK spinal mesh to the prosthetic device;

FIG. 14 is a view of a portion of a prosthetic disc and PEEK spinal mesh in accordance with the present principles particularly illustrating another manner of attachment of the PEEK spinal mesh to the prosthetic device;

FIG. 15 is a front (anterior) view of a prosthetic disc having PEEK spinal mesh integrally molded with the viscous body of the prosthetic disc;

FIG. 16 is a side (medial) view of the prosthetic disc of FIG. 15;

FIG. 17 is an enlarged perspective view of a portion of the spine of FIG. 1 showing a use of the present PEEK spinal mesh with respect to the annulus fibrosus of a spinal disc;

FIG. 18 is an enlarged perspective view of another portion of the spine of FIG. 1 showing another use of the present PEEK spinal mesh particularly with respect to an annulus fibrosus of a spinal disc;

FIG. 19 is an enlarged perspective view of the portion of the spine of FIG. 18 showing yet another use of the present PEEK spinal mesh particularly with respect to an annulus fibrosus and portions of adjacent vertebrae of the spine;

FIG. 20 is an enlarged side (medial) view of the portion of the spine of FIG. 1 showing a spinous process band fashioned in accordance with the present principles and disposed about adjacent spinous processes and used in conjunction with the present PEEK spinal mesh;

FIG. 21 is a rear or anterior view of the portion of the spine of FIG. 20 showing the present PEEK band installed on adjacent spinous processes; and

FIG. 22 is a perspective view of an embodiment of a PEEK spinal mesh applicator fashioned in accordance with the present principles.

Corresponding reference characters indicate corresponding parts throughout the several views. Although the drawings represent embodiments of the invention, the drawings are not necessarily to scale and certain features may be exaggerated in order to better illustrate and explain the present invention. The exemplifications set out herein illustrate embodiments of the invention, and such exemplifications are not to be construed as limiting the scope of the invention in any manner.

### DETAILED DESCRIPTION OF THE INVENTION

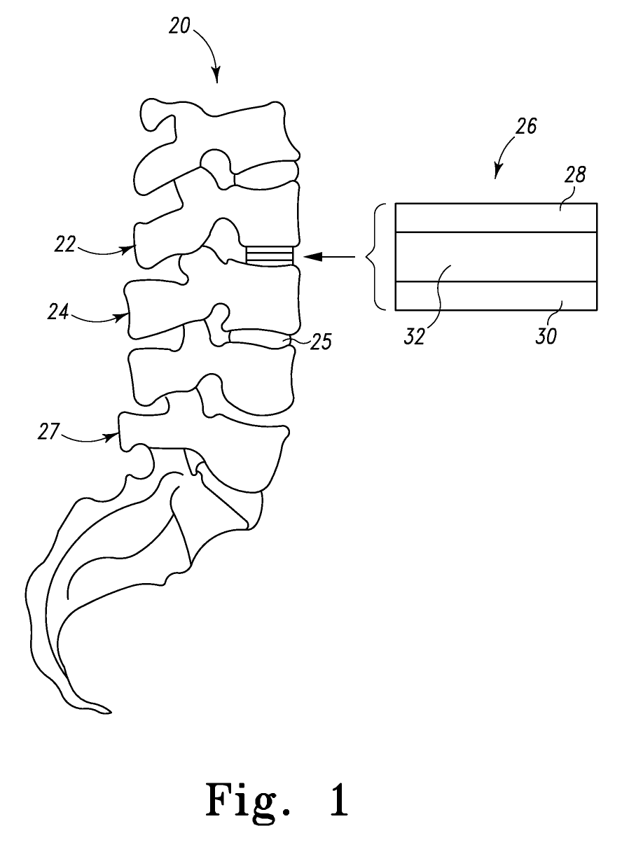

Referring FIG. 1 illustrates a lower portion of a typical human spinal column 20 on which the present invention is used and/or onto which the present invention is applied. Two adjacent vertebrae, labeled 22 and 24, are enumerated as representative of any two adjacent vertebrae regardless of spinal location. A prosthetic or artificial spinal disc 26 is shown provided between the adjacent vertebrae 22, 24 as a replacement for a removed spinal disc. Because the present invention works with other types of spinal prosthetics or devices, the prosthetic spinal disc 26 should be considered as only representative of a type of inter-vertebral body. It should be appreciated that while the various figures depict an artificial disc, the present invention is usable relative to natural disc or disc material. Therefore, the various descriptions are applicable to both instances except where specifically limited.

FIG. 1 thus illustrates an inter-vertebral spinal disc prosthesis, e.g. artificial or prosthetic spinal disc 26. The spinal disc 26 is illustrated as implanted between adjacent upper and lower vertebrae 22 and 24 of the human spinal column 20. The spinal disc 26 has been implanted in a manner such as in known in the art. The vertebrae 22 and 24 have portions that face anteriorly (to the right as viewed in FIG. 1) and portions that face posteriorly (to the left as viewed in FIG. 1). The various ligaments of the spinal column 20 are not shown, but of course include the anterior longitudinal ligament, the posterior longitudinal ligament, and the ligamentum flavum.

The disc 26 is representative of current artificial disc technology. Disc 26 comprises a first or upper rigid plate 28, a second or lower rigid plate 30, and a core 32 interposed between and adhered to the two rigid plates. The core 32 may be a hydro-gel or the like. The terms “upper” and “lower” are used herein with reference to the orientation of the disc 26 when it is implanted into the human body as illustrated in FIG. 1 for clarity.

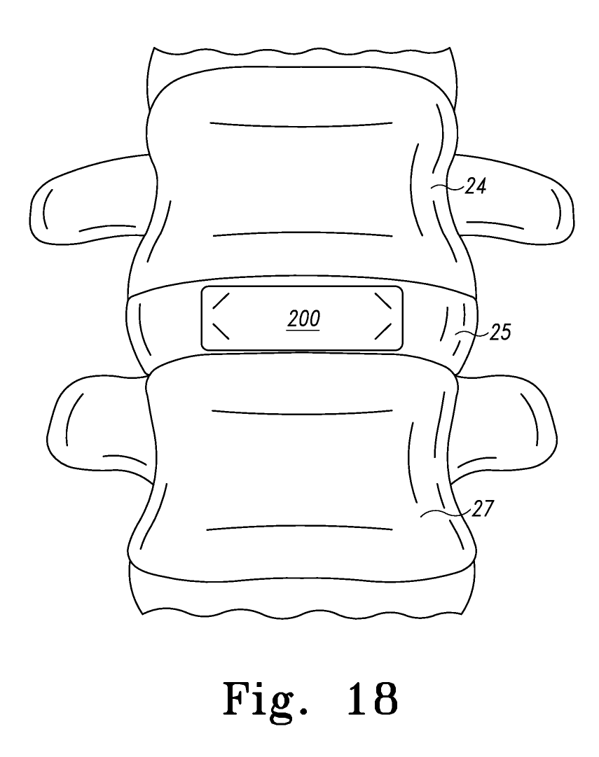

FIG. 1 also shows another vertebra 27 that is below vertebra 24. A natural spinal disc 25 is positioned between vertebrae 27 and 24. The present invention is applicable to the spinal area still having a natural disc (i.e. disc 25) as well as spinal areas having an artificial or prosthetic disc (i.e. disc 26) such as is shown in FIGS. 18-21. The various ligaments of the spine are not illustrated in FIG. 1.

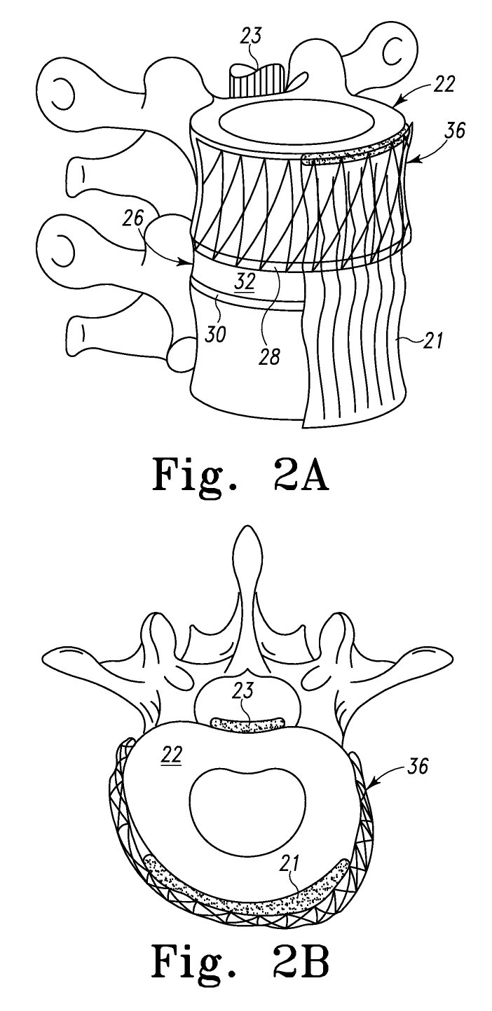

In FIGS. 2A and 2B there is illustrated the vertebrae 22 and 24 of FIG. 1. It should be appreciated that the proportions of the various components or parts hereof (with respect to all figures) as well as the proportions of the components relative to one another are not necessarily exact. In accordance with the principles of the subject invention, in one form, a mesh, covering, net, netting, web, webbing, bag, shroud or the like 36 (collectively herein, mesh) is provided, particularly shown with respect to the surgical area of the spinal implant. In one form, the present mesh extends from an upper end of the disc 26 and essentially if not completely entirely encapsulates the upper vertebra 22 and a portion of the anterior longitudinal ligament 21. The lower end of the PEEK mesh 36 is secured to the disc 25 while the upper end of the PEEK mesh 36 is secured to the upper end of the vertebra 22.

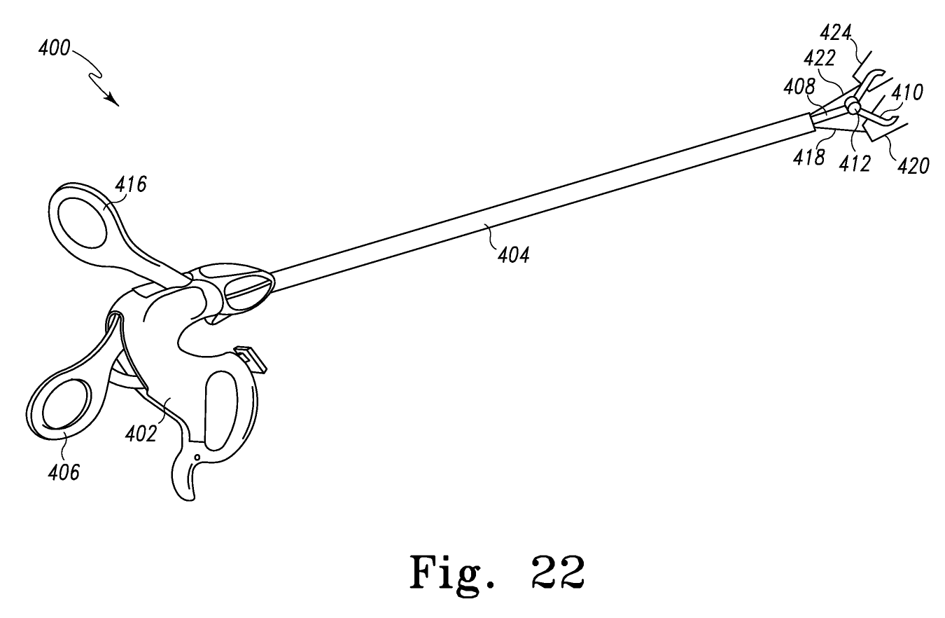

The present invention is thus a bio-compatible mesh for use in spinal applications, such as during spinal surgery, to cover, shroud and/or encapsulate at least a portion of one or more vertebrae and/or inter-vertebral devices. Preferably, but not necessarily, the present bio-compatible mesh is formed of PEEK (polyetheretherketone). A delivery instrument for the present PEEK mesh is also provided (see FIG. 22) that places the PEEK mesh onto spinal surfaces (e.g. vertebra and implants) and then applies fasteners to the PEEK mesh and the spinal surfaces for holding the PEEK mesh to the spinal surfaces. The present PEEK mesh delivery instrument is preferably, but not necessarily, a laproscopic delivery instrument. The delivery instrument provides a method of installing the PEEK mesh.

As best seen in FIG. 2B, the PEEK mesh 36 extends about the upper portion (e.g. the plate 28 and/or the upper portion of the core 32) of the disc 26 and the vertebra 22 essentially from one side of the vertebra 22 to the opposite side of the vertebra 22. Particularly, the PEEK mesh 36 extends essentially around the upper portion of the disc 26 and the vertebra 22 from the left pedicle to the right pedicle of the vertebra 22 as viewed in FIGS. 2A and 2B. The lower portion of the PEEK mesh 36 is secured to or formed integrally with, the disc 26. If the lower portion of the PEEK mesh 36 is secured to the disc 26, the lower portion of the PEEK mesh 36 may be secured to either the plate 28 or the core 32. The upper portion of the PEEK mesh 36 is secured to the exposed vertebra 22 and may additionally or alternatively be secured to the anterior longitudinal ligament 21.

The PEEK mesh 36 may be formed of a single layer or multiple layers. Multiple layers may be of the same material and/or design, or may be different materials. The mesh may encapsulate an agent for aiding in the adhesion of the mesh to its intended host(s) and/or for a medicinal purpose such as described in the above summary.

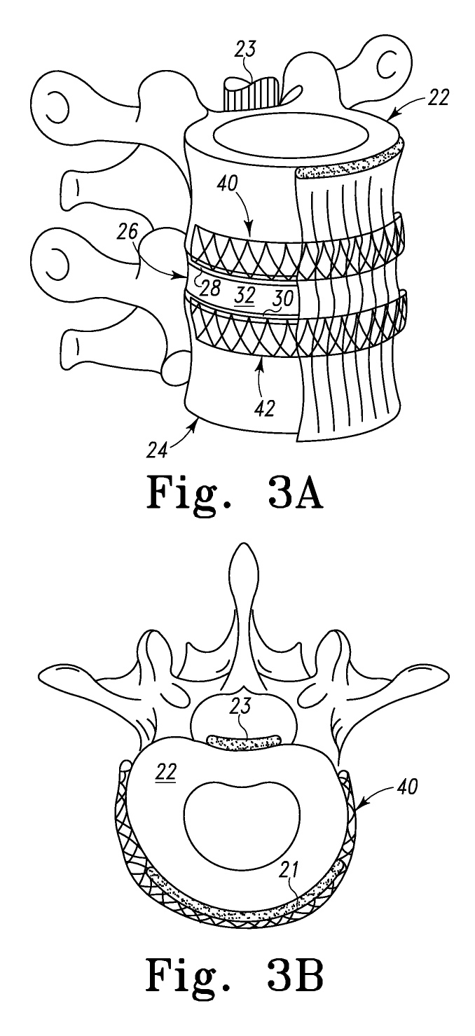

In FIGS. 3A and 3B there is illustrated the vertebrae 22 and 24 of FIG. 1 between which the artificial disc 26 has been implanted. In accordance with the principles of the subject invention, in one form, an upper PEEK mesh is provided in the surgical area of the spinal implant. The upper PEEK mesh 40 extends from an upper end (e.g. plate 28 or an upper portion of the core 32) of the disc 26 and is secured to the vertebra 22 at a lower or middle portion thereof. The upper PEEK mesh 40 thus covers or encapsulates an upper portion of the disc 26 and a middle to lower portion of the vertebra 22 as well as a portion of the anterior longitudinal ligament 21. A lower PEEK mesh 42 is also provided that extends from a lower end (e.g. plate 30 or an upper portion of the core 32) of the disc 26 and is secured to the vertebra at an upper or middle portion thereof. The lower PEEK mesh 42 thus covers or encapsulates a lower portion of the disc 26 and a middle to upper portion of the vertebra 24 as well as a portion of the ligament 21. The upper and lower PEEK meshes 40 and 42 each has like characteristics and/or properties as the PEEK mesh 36.

As best seen in FIG. 3B, the PEEK mesh 40 extends about the upper portion (e.g. the plate 28 and/or the upper portion of the core 32) of the disc 26 and a portion of the lower vertebra 22 essentially from one side of the vertebra 22 to the opposite side of the vertebra 22. Particularly, the PEEK mesh 40 extends essentially around the upper portion of the disc 26 and a lower portion of the vertebra 22 from the left pedicle to the right pedicle of the vertebra 22 as viewed in FIGS. 3A and 3B.

The lower portion of the PEEK mesh 40 is secured to or formed integrally with, the disc 26. If the lower portion of the PEEK mesh 40 is secured to the disc 26, the lower portion of the PEEK mesh 40 may be secured to either the plate 28 or the core 32. The upper portion of the PEEK mesh 40 is secured to the exposed vertebra 22 and may additionally or alternatively be secured to the anterior longitudinal ligament 21.

While not seen in FIG. 3B, but in like manner to the PEEK mesh 40, the PEEK mesh 42 extends around the lower portion (e.g. the plate 30 and/or the lower portion of the core 32) of the disc 26 and an upper portion of the vertebra 24 essentially from one side of the vertebra 24 to the opposite side of the vertebra 24. Particularly, the PEEK mesh 42 extends essentially around the lower portion of the disc 26 and an upper portion of the vertebra 24 from the left pedicle to the right pedicle of the vertebra 24 as viewed in FIGS. 3A and 3B.

The upper portion of the PEEK mesh 42 is secured to or formed integrally with, the disc 26. If the upper portion of the PEEK mesh 42 is secured to the disc 26, the upper portion of the PEEK mesh 42 may be secured to either the plate 28 or the core 32. The lower portion of the PEEK mesh 42 is secured to the exposed vertebra 24 and may additionally or alternatively be secured to the anterior longitudinal ligament 21.

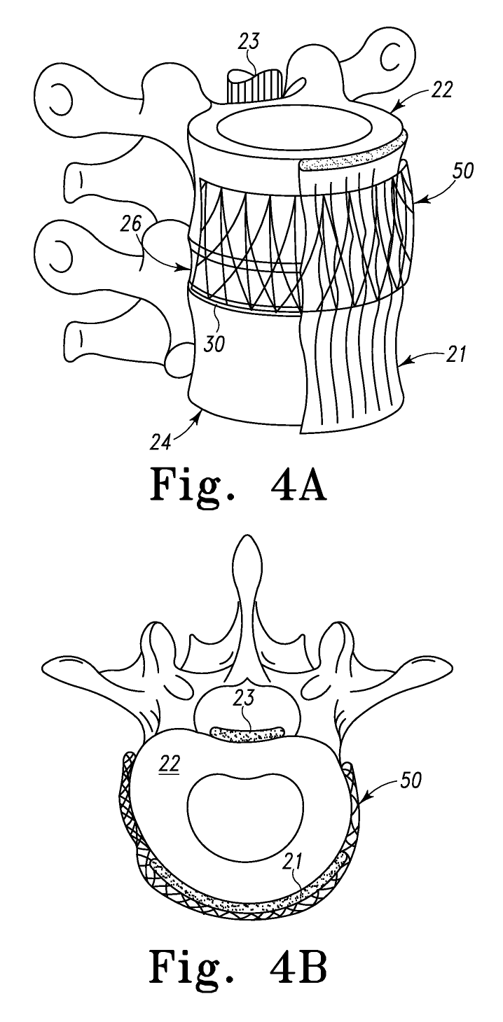

In FIGS. 4A and 4B, there is illustrated the vertebrae 22 and 24 of FIG. 1 between which the artificial disc 26 has been implanted. In accordance with the principles of the subject invention, in one form, a PEEK mesh is provided in the surgical area of the spinal implant. The PEEK mesh 50 extends from a lower upper end (e.g. plate 30 or a lower portion of the core 32) of the disc 26 to an upper portion of the vertebra and therearound. The PEEK mesh 50 is secured to the vertebra 22 at the upper portion thereof. The PEEK mesh 50 thus covers or encapsulates a portion of the disc 26 on both sides of the spine, an upper portion of the vertebra 22, as well as a portion of the anterior longitudinal ligament 21. The PEEK mesh 50 has like characteristics and/or properties as the PEEK mesh 36.

As best seen in FIG. 4B, the PEEK mesh 50 extends about the lower portion (e.g. the plate 30 and/or the lower portion of the core 32) of the disc 26 and most of the vertebra 22 essentially from one side of the vertebra 22 to the opposite side of the vertebra 22. Particularly, the PEEK mesh 50 extends essentially around the disc 26 and a most of the vertebra 22 from the left pedicle to the right pedicle of the vertebra 22 as viewed in FIGS. 4A and 4B.

The lower portion of the PEEK mesh 50 is secured to or formed integrally with, the disc 26. If the lower portion of the PEEK mesh 50 is secured to the disc 26, the lower portion of the PEEK mesh 50 may be secured to either the plate 30 or the core 32. The upper portion of the PEEK mesh 40 is secured to the exposed vertebra 22 and may additionally or alternatively be secured to the anterior longitudinal ligament 21.

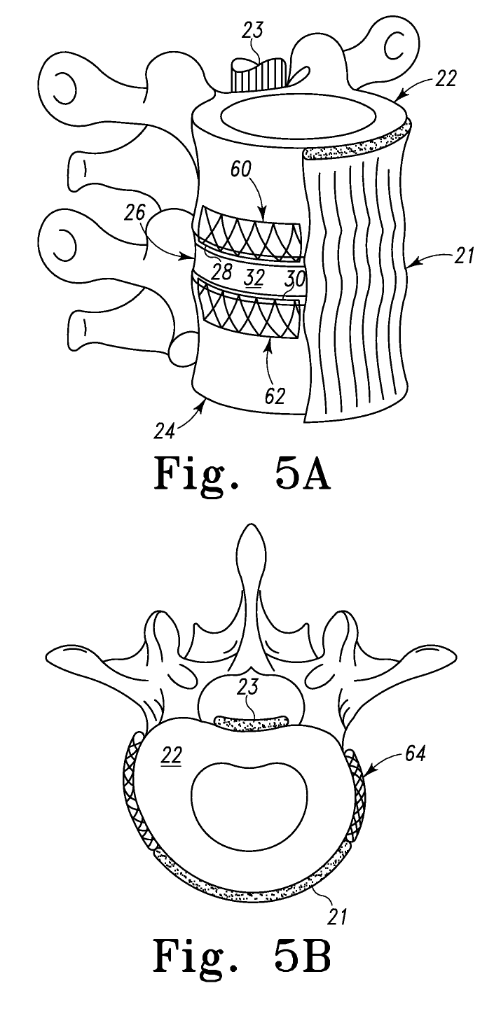

In FIGS. 5A and 5B there is illustrated the vertebrae 22 and 24 of FIG. 1 between which the artificial disc 26 has been implanted. In accordance with the principles of the subject invention, in one form, a first upper PEEK mesh is provided in the surgical area of the spinal implant. The first upper PEEK mesh 60 extends from an upper end (e.g. plate 28 and/or an upper portion of the core 32) of one side of the disc 26 and is secured to the vertebra 22 at a lower or middle portion thereof. The first upper PEEK mesh 60 covers or encapsulates an upper side portion of the disc 26 and a middle to lower side portion of the vertebra 22 between the anterior longitudinal ligament 21 and the adjacent pedicle of the vertebra 22.

A first lower PEEK mesh 62 is provided that extends from a lower end (e.g. plate 30 and/or a lower portion of the core 32) of a side of the disc 26 and is secured to the vertebra 24 at a middle to upper portion thereof. The lower PEEK mesh 62 covers or encapsulates a lower side portion of the disc 26 and a middle to upper side portion of the vertebra 24 between the anterior longitudinal ligament 21 and the adjacent pedicle of the vertebra 24.

As seen in FIG. 5B, a second upper PEEK mesh 64 is further provided that extends from an upper end (e.g. plate 28 and/or an upper portion of the core 32) of another side of the disc 26 (opposite PEEK mesh 60) and is secured to the vertebra 22 at a lower or middle portion thereof. The second upper PEEK mesh 64 covers or encapsulates an upper side portion of the disc 26 and a middle to lower side portion of the vertebra 22 radially between the anterior longitudinal ligament 21 and the adjacent pedicle of the vertebra 22.

While not seen in FIGS. 5A and 5B, a second lower PEEK mesh 66 is provided that extends from a lower end (e.g. plate 30 and/or a lower portion of the core 32) of another side of the disc 26 and is secured to the vertebra 24 at a middle to upper portion thereof. The lower PEEK mesh 66 covers or encapsulates a lower side portion of the disc 26 and a middle to upper side portion of the vertebra 24 between the anterior longitudinal ligament 21 and the adjacent pedicle of the vertebra 24. It should be appreciated that the PEEK mesh bags 60, 62, 64 and 66 each has like characteristics and/or properties as the PEEK mesh 36.

The lower portions of the PEEK meshes 60 and 64 are secured to or formed integrally with, the disc 26. If the lower portions of the PEEK meshes 60 and 64 are secured to the disc 26, the lower portions of the PEEK meshes 60 and 64 may be secured to the plate 28 and/or the core 32. The upper portion of the PEEK meshes 60 and 64 are secured to the exposed vertebra 22.

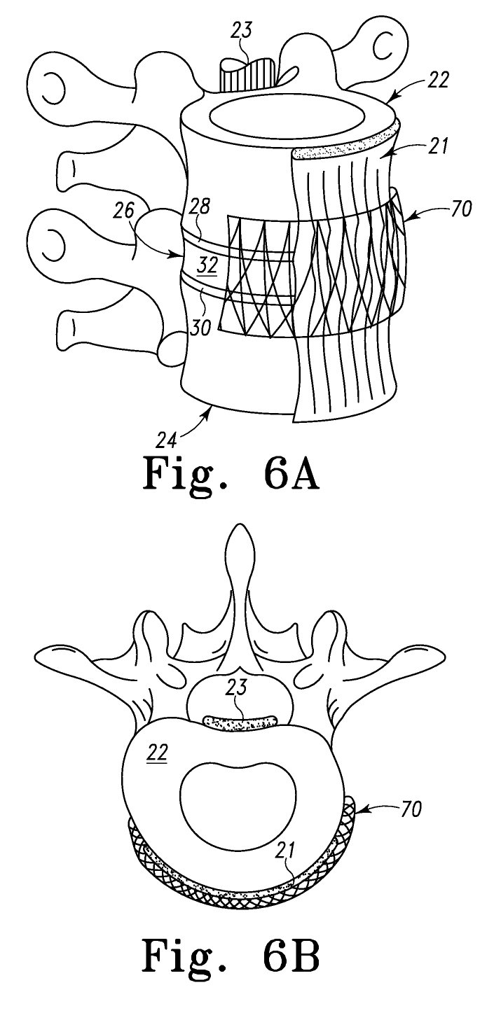

In FIGS. 6A and 6B there is illustrated the vertebrae 22 and 24 of FIG. 1 between which the artificial disc 26 has been implanted. In accordance with the principles of the subject invention, in one form, a single PEEK mesh 70 is provided in the surgical area of the spinal implant. In one dimension, the PEEK mesh 70 extends from a middle to an upper portion of the vertebra 24 to a middle to lower portion of the vertebra 22 around the two vertebrae 22, 24. In a radial dimension, the PEEK mesh 70 extends about the vertebrae 22, 24 from an approximately middle area between the anterior longitudinal ligament and the vertebra pedicle on both sides thereof. The PEEK mesh 70 thus covers or encapsulates only a portion of the disc 26 on both sides of the spine, as well as an upper portion of the vertebra 22 and a portion of the anterior longitudinal ligament 21. The PEEK mesh 70 has like characteristics and/or properties as the PEEK mesh 36. The upper and lower portions of the PEEK mesh 70 are secured to the exposed vertebra 22 and 24.

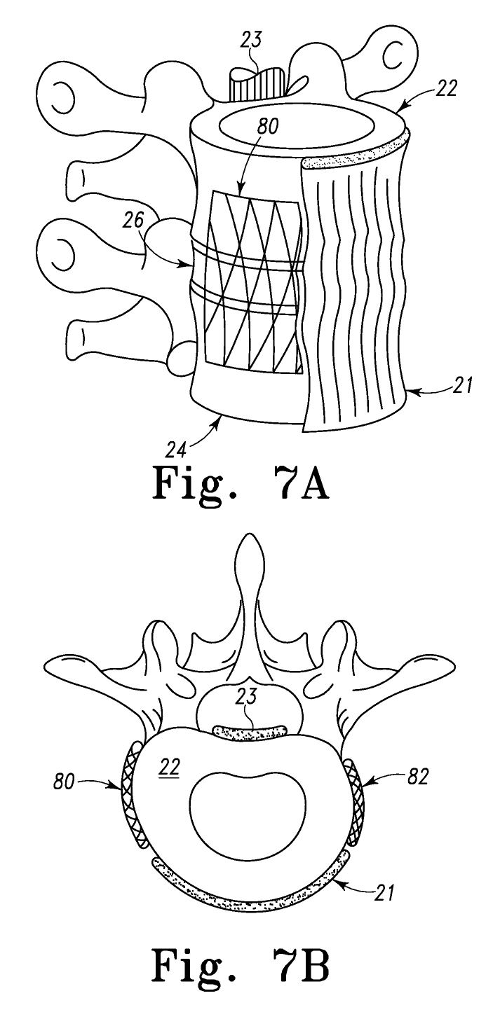

In FIGS. 7A and 7B there is illustrated the vertebrae 22 and 24 of FIG. 1 between which the artificial disc 26 has been implanted. In accordance with the principles of the subject invention, in one form, a first PEEK mesh 80 and a second PEEK mesh 82 is provided in the surgical area of the spinal implant. The first PEEK mesh 80 is disposed on one side of the vertebrae 22 and 24 over the portion of the disc 26 that is exposed between the adjacent side of the anterior longitudinal ligament 21 and the pedicles. The second PEEK mesh 82 is disposed on another side of the vertebrae 22, 24 opposite the side of the site of the first PEEK mesh 80. The second PEEK mesh 82 is disposed over the portion of the disc 26 that is exposed between the adjacent side of the anterior longitudinal ligament 21 and the pedicles.

In one dimension, first the PEEK mesh 80 extends from a lower to a middle portion of the vertebra 24 and from a middle to an upper portion of the vertebra 22. In a radial dimension, the first PEEK mesh 80 extends about the vertebrae 22, 24 from the pedicles thereof to proximate the side of the anterior longitudinal ligament 21. The first PEEK mesh 80 thus covers or encapsulates the disc 26 on one side of the vertebrae 22, 24. The first PEEK mesh 80 has like characteristics and/or properties as the PEEK mesh 36. The upper and lower portions of the first PEEK mesh 80 are secured to the exposed vertebra 22 and 24.

In one dimension, second the PEEK mesh 82 extends from a lower to a middle portion of the vertebra 24 and from a middle to an upper portion of the vertebra 22 in like manner to the first PEEK mesh 80. In a radial dimension, the second PEEK mesh 82 extends about the vertebrae 22, 24 from the pedicles thereof to proximate the side of the anterior longitudinal ligament 21. The second PEEK mesh 82 thus covers or encapsulates the disc 26 on one side of the vertebrae 22, 24 but opposite the first PEEK mesh 80. The second PEEK mesh 82 has like characteristics and/or properties as the PEEK mesh 36. The upper and lower portions of the second PEEK mesh 82 are secured to the exposed vertebra 22 and 24.

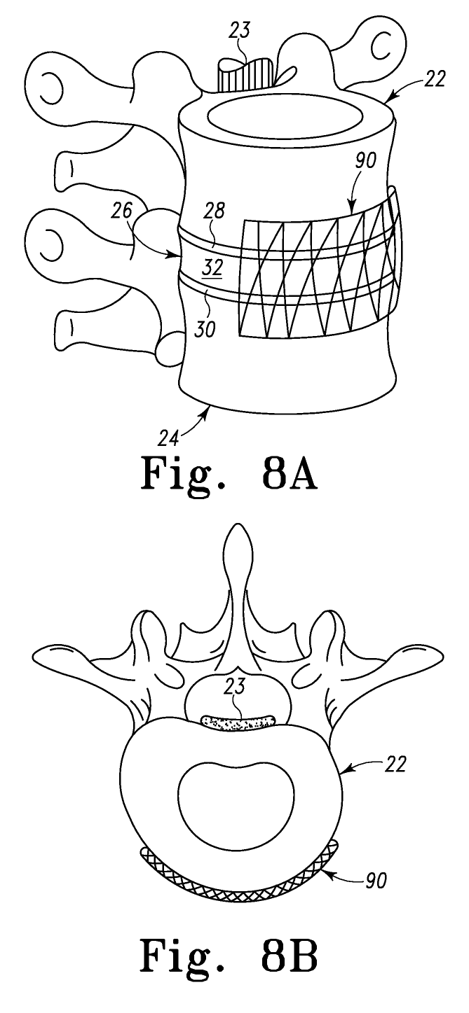

In FIGS. 8A and 8B there is illustrated the vertebrae 22 and 24 of FIG. 1 between which the artificial disc 26 has been implanted. Additionally, the anterior longitudinal ligament (ALL) 21 is not shown in FIGS. 8A and 8B representing various states of the ALL (e.g. resected, partial or wholly removed, injured, etc.). In accordance with the principles of the subject invention, in one form, a PEEK mesh 90 is provided in the surgical area of the spinal implant. The PEEK mesh 90 is on the anterior portion of the vertebrae 22 and 24 and extends in one dimension from an approximate middle portion of the vertebra 24 to an approximate middle portion of the vertebra 22. In a radial direction, the PEEK mesh 90 extends about the vertebrae 22, 24 in like size or larger than the original ALL. The upper and lower portions of the PEEK mesh 90 are secured to the exposed vertebra 22 and 24.

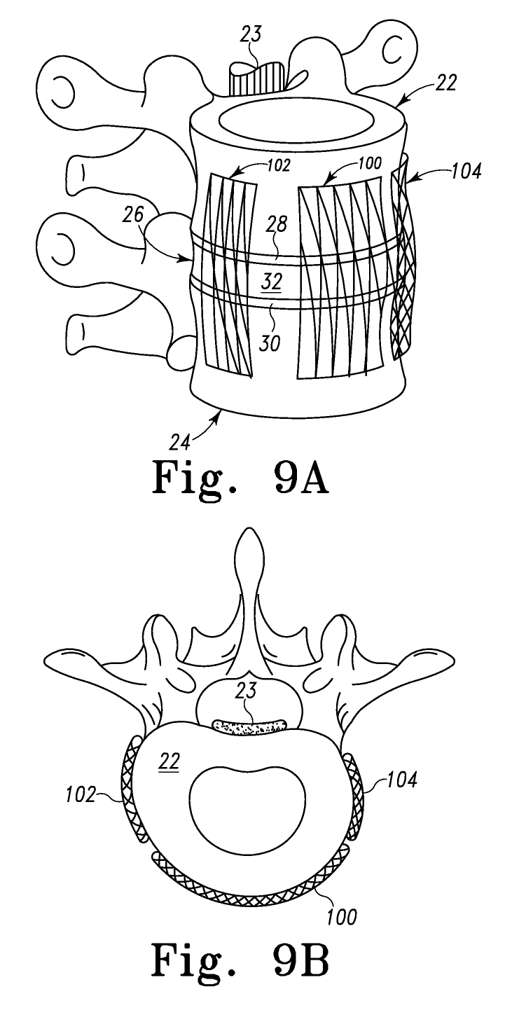

In FIGS. 9A and 9B there is illustrated the vertebrae 22 and 24 of FIG. 1 between which the artificial disc 26 has been implanted. Additionally, the anterior longitudinal ligament (ALL) 21 is not shown in FIGS. 9A and 9B in like reason as FIGS. 8A and 8B. In accordance with the principles of the subject invention, in one form, first, second and third PEEK meshes 100, 102 and 104 are provided in the surgical area of the spinal implant.

The first PEEK mesh 100 is on the anterior portion of the vertebrae 22 and 24 and extends in one dimension from an approximate lower portion of the vertebra 24 to an approximate upper portion of the vertebra 22. In a radial direction, the first PEEK mesh 100 extends about the vertebrae 22, 24 in an approximate like size than the original ALL. The upper and lower portions of the first PEEK mesh 100 are secured to the exposed vertebra 22 and 24. The first PEEK mesh 100 covers or encapsulate the anterior portion of the disc 26.

The second PEEK mesh 102 is on one side of the vertebrae 22 and 24 to cover or encapsulate a first side of the disc 26. The second PEEK mesh 102 extends in one dimension from a lower to middle portion of the vertebra 24 to the middle or upper portion of the vertebra 22. In a radial direction, the second PEEK mesh 102 extends about a middle portion of the area between the pedicles of the vertebrae 22, 24 and the end of the ALL. The upper and lower portions of the second PEEK mesh 102 are secured to the exposed vertebra 22 and 24.

The third PEEK mesh 104 is on another side of the vertebrae 22 and 24 opposite the second PEEK mesh 102 so as to cover or encapsulate a second side of the disc 26. The third PEEK mesh 104 extends in one dimension from a lower to middle portion of the vertebra 24 to the middle or upper portion of the vertebra 22. In a radial direction, the third PEEK mesh 104 extends about a middle portion of the area between the pedicles of the vertebrae 22, 24 and the end of the ALL. The upper and lower portions of the third PEEK mesh 104 are secured to the exposed vertebra 22 and 24.

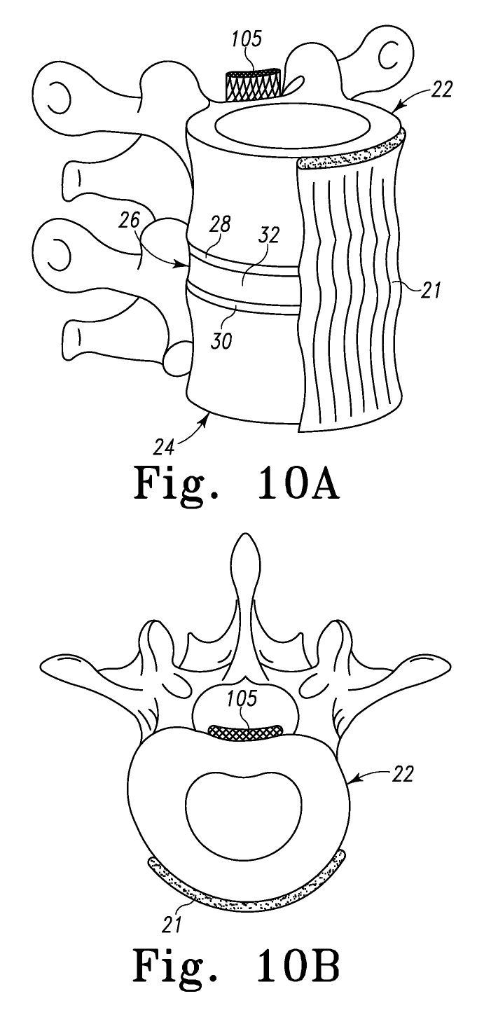

In FIGS. 10A and 10B there is illustrated the vertebrae 22 and 24 of FIG. 1 between which the artificial disc 26 has been implanted. A portion of the posterior longitudinal ligament (PLL) 23 has been provided with PEEK mesh 105 to represent the use of the present PEEK mesh with and/or for spinal ligament or ligament material. The PEEK mesh portion 105 may be the PLL PEEK mesh wrapped or covered to a various degree.

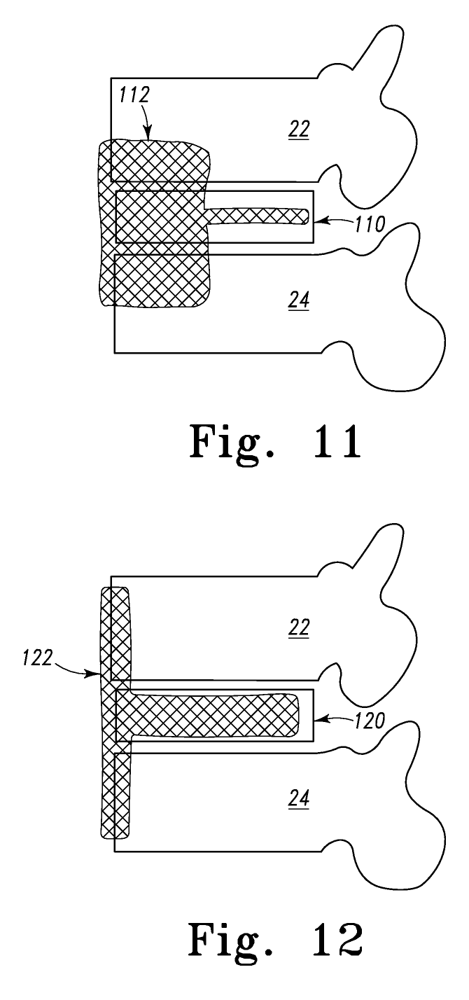

In FIG. 11 there is depicted a side (medial) view of the vertebrae 22 and 24 between which has been implanted a spinal disc 110. A PEEK mesh 112 has been provided that extends about the ALL and covers an anterior portion of the implant or spinal opening between the pedicles and the ALL. The PEEK mesh 112 includes first and second elongated tabs or strips of PEEK mesh (of which only one strip 114 can be seen) that extend from the sides of the main body 116 of the PEEK mesh 112. Each strip is attached or secured to the disc 26 as described below.

In FIG. 12 there is depicted a side (medial) view of the vertebrae 22 and 24 between which has been implanted a spinal disc 120. A PEEK mesh 122 has been provided that extends about the front portion of the ALL. The PEEK mesh 122 includes first and second elongated tabs or strips of PEEK mesh (of which only one strip 124 can be seen) that extend from the sides of the main body 126 of the PEEK mesh 122. Each strip is attached or secured to the disc 26 as described below.

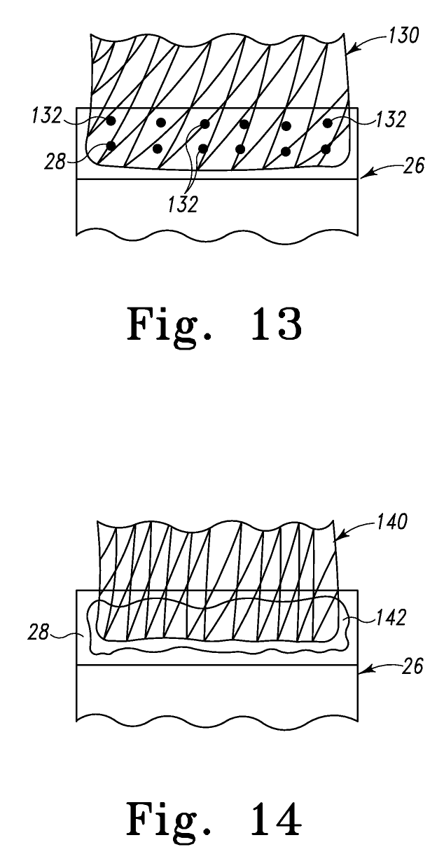

FIGS. 13 and 14 illustrate exemplary, but not exclusive, manners of securing or attaching a PEEK mesh as provided herein to a disc. It should be appreciated that while the manner of attachment is illustrated with respect to a disc, they are applicable to the vertebrae as well. In FIG. 13, a portion of a PEEK mesh 130 is shown relative to the artificial disc 26 and specifically with respect to the plate 28 of the disc 26. A plurality of staples, nails or other attachment devices 132 are used to secure the PEEK mesh 130 to the plate. In FIG. 14, a portion of a PEEK mesh 140 is shown relative to the artificial disc 26 and specifically with respect to the plate 28 of the disc 26. A bio-compatible adhesive 142 is used to secure the PEEK mesh 140 to the disc 26. Of course, other methods or manners of attachment may be used and are contemplated.

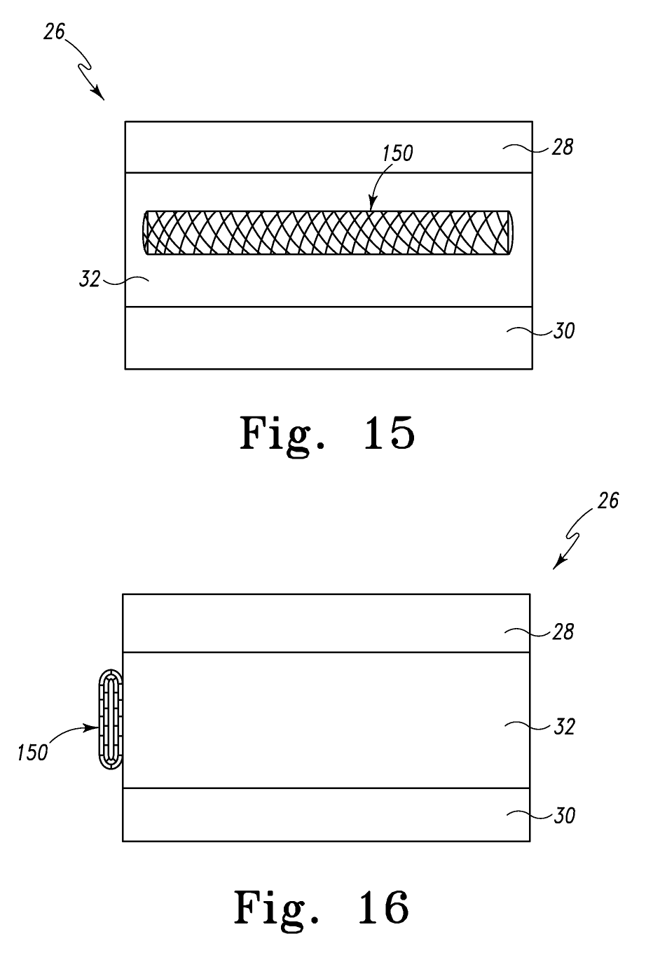

Referring to FIGS. 15 and 16 the prosthetic spinal disc 26 is shown having a PEEK mesh bag or wrapped amount 150 integrally formed with the core 32 of the disc 26. The PEEK mesh bag 150 is shown in an un-deployed state. The PEEK mesh bag 150 is ready to be unwrapped, placed and secured or attached to the vertebrae as appropriate. While only one such PEEK mesh bag is shown, the disc 26 may have two or more PEEK mesh bags. In this manner, once the disc 26 is implanted, the PEEK mesh 150 may be installed as appropriate.

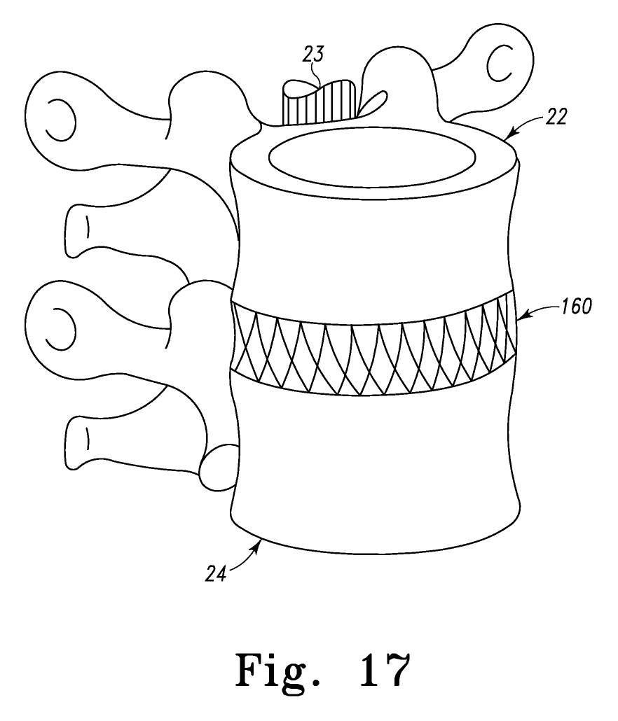

FIG. 17 illustrates the use of PEEK mesh 160 as a supplement to the annulus fibrosus of a patient's disc. The PEEK mesh 160 is shown in FIG. 17 as encompassing the circumference of the disc. The PEEK mesh 160 may be provided for only a portion of the natural annulus fibrosus.

FIG. 18 illustrates the use of PEEK mesh 200 as a supplement to the annulus fibrosis of a patient's disc with respect to the vertebral column of FIG. 1 and, particularly, to the disc 25 situated between vertebrae 24 and 27. The PEEK mesh 200 is attached to the disc 25 via staples provided by the PEEK mesh delivery and fixation instrument 400 (see FIG. 22). After delivery of the PEEK mesh 200 to the spinal site or surfaces by the PEEK mesh delivery and fixation instrument 400. Four (4) staples are shown at the corners of the PEEK mesh 200 that attach the PEEK mesh 200 to the disc 25.

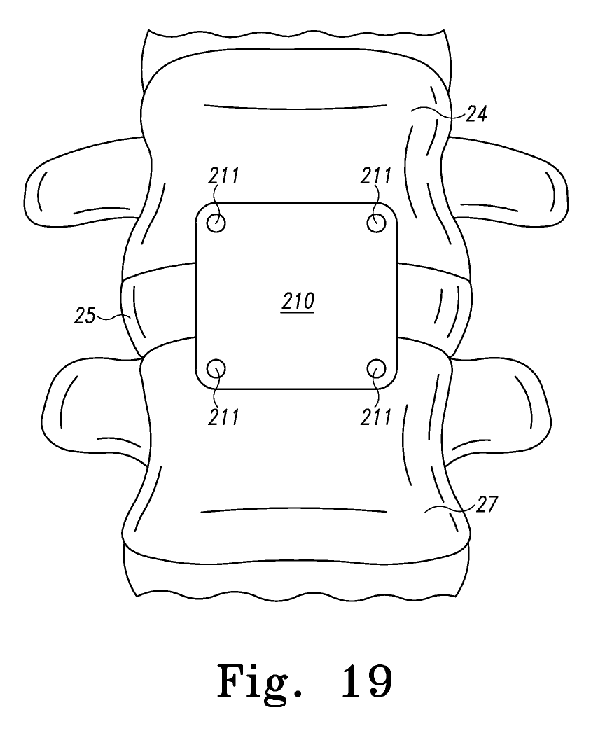

FIG. 19 illustrates the use of PEEK mesh 210 as bracing against the annulus fibrosis of a patient's disc by attachment to upper and lower vertebrae 24, 27 with respect to the vertebral column of FIG. 1. The PEEK mesh 210 has four attachment points 211 for attaching the PEEK mesh 210 to the upper and lower vertebrae 24, 27 via the PEEK mesh delivery and fixation instrument 400 (see FIG. 22) after delivery of the PEEK mesh 210 to the spinal sites or surfaces by the PEEK mesh delivery and fixation instrument 400.

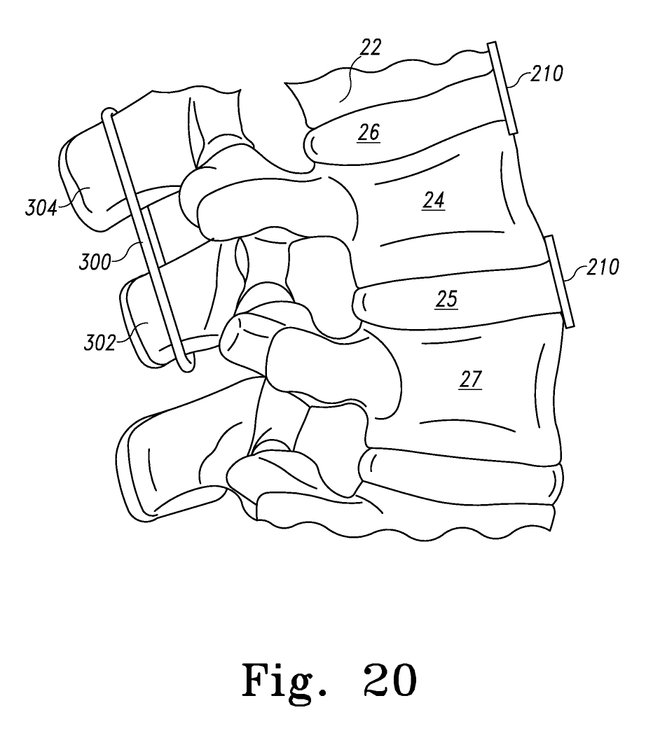

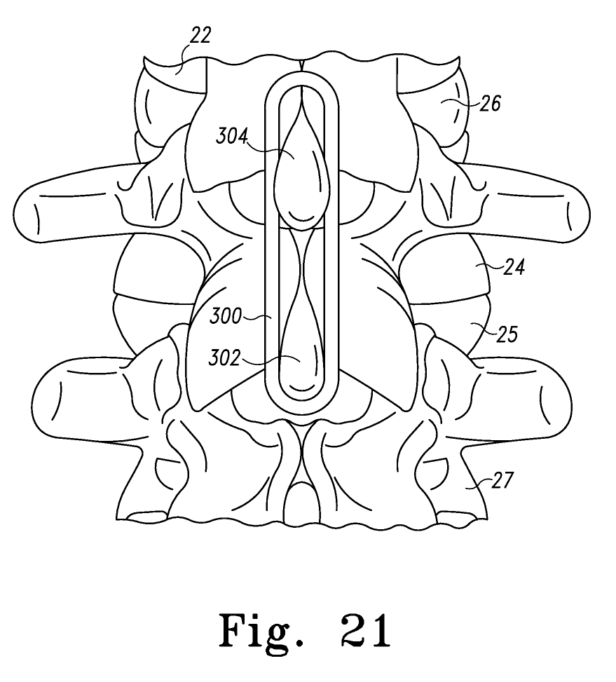

FIGS. 20 and 21 illustrate the use of a spinous process band or ring 300 used in conjunction with the PEEK mesh (such as the PEEK mesh 210 shown in FIG. 20). The spinous process band 30 is preferably formed of PEEK (polyetheretherketone) as well, but may be formed of other biocompatible materials such as described herein. The spinous process band 30 is sized to be received around two adjacent spinous processes, 302, 304. This limits the amount of anterior compression on and of the associated vertebrae (here, vertebrae 22, 24). This helps to maintain the PEEK mesh in place while the healing process occurs.

FIG. 22 depicts an exemplary instrument 400 for delivering, applying and/or attaching mesh as described herein and particularly, but not necessarily, PEEK mesh. The instrument 400 is preferably, but not necessarily, a minimally invasive instrument for providing PEEK mesh and fastenings means minimally invasively (e.g. laproscopically). The PEEK mesh applicator 400 includes a handle 402 to which is attached a shaft 404. The handle 402 is preferably ergonomically fashioned and includes a first trigger 406 and a second trigger 416. The first trigger 406 operates a mesh application portion of the applicator 400 to apply the mesh to the spinal surface. In another embodiment not shown, the triggers are combined such that mesh application and fastening are accomplished simultaneously. The applicator 400 can apply then fasten the mesh on a particular spinal site or it may apply and fasten the mesh essentially simultaneously.

The first trigger 406 is coupled to a shaft 408 that controls opening and closing of fingers 410 that are distal to the handle 402. The shaft 408 extends axially within the shaft 404. The fingers 410 are pivotally coupled to the control shaft 408 via a pivot 412 and serve to hold, spread and apply PEEK mesh such as described herein, to a spinal surface. Movement of the first trigger 406 relative to the handle 402 opens and closes the fingers 410 appropriately. It should be appreciated that while only two (2) fingers 410 are shown, more fingers may be provided such as four (4) that can hold an amount of PEEK mesh and spread the PEEK mesh onto the spinal surface. Movement of the first trigger 406 moves the fingers 410. The shaft 408 may or may not move axially with respect to the shaft (tube) 404.

The second trigger 416 of the applicator 400 operates an attachment portion that controls the application of fasteners and/or fastening means to the applied PEEK mesh so as to attach the applied PEEK mesh to the spinal surface. In the embodiment shown in FIG. 22, the attachment portion has two (2) staples 420, 424. The staples are preferably, but not necessarily, barbed bone anchors. There can be two (2) staples that are U-shaped such as shown, four (4) staples that go in diverging directions or other configurations. Movement of the second trigger 416 relative to the handle 402 moves and applies the staples 420, 424 or other adhesion means to the mesh and spinal surfaces to attach the mesh to the spinal surfaces.

Of course, other configurations are contemplated for a mesh applicator that provides a mesh delivery and attachment device in a single instrument such as the present mesh applicator 400.

It should be appreciated that the above figures and descriptions are only exemplary of the many mesh configuration and dimensions possible in accordance with the present principles.

In accordance with another aspect of the present invention, methods for implanting the various meshes in the configurations depicted in the figures are provided. In the case of non-integrally formed mesh, the mesh is implanted or installed after prosthetic disc implantation. In the case of mesh integrally formed with the disc, the mesh is unwrapped, extended about the appropriate site and secured after disc implantation.

While this invention has been described as having a preferred design, the present invention can be further modified within the spirit and scope of this disclosure. This application is therefore intended to cover any variations, uses, of adaptations of the invention using its general principles. Further, this application is intended to cover such departures from the present disclosure as come within known or customary practice in the art to which this invention pertains.

What is claimed is:

1. A surgical treatment for the spine of a patient comprising the steps of:
incising the patient proximate vertebrae of the spine desired to be treated;
introducing a configured sheet of a biocompatible mesh to the vertebrae desired to be treated through an incision created by the step of incising; and
attaching the configured sheet to an area about the vertebrae desired to be treated.

2. The method of claim 1, wherein incising the patient is performed through minimally invasive surgery.

3. The method of claim 1, wherein the configured sheet of a biocompatible mesh is comprised of PEEK.

4. The method of claim 1, wherein the area about the vertebrae desired to be treated covers a site of a spinal surgical procedure.

5. The method of claim 1, wherein the area about the vertebrae desired to be treated extends from an upper vertebra to a lower vertebra of the vertebrae desired to be treated.

6. The method of claim 1, wherein the area about the vertebrae desired to be treated extends radially about the upper and lower vertebrae.

7. The method of claim 1, wherein the configured sheet is attached to the area about the vertebrae desired to be treated by one or more of staples, an adhesive, screws, wiring, and tying.

8. The method of claim 1, wherein the configured sheet is introduced to the vertebrae desired to be treated while rolled up.

9. The method of claim 1, further comprising the steps of:
introducing a spinous process band through the incision; and
placing the spinous process band around the spinous processes of upper and lower vertebrae of the vertebrae desired to be treated.

10. The method of claim 9, wherein the spinous process band is formed of PEEK.

11. A spinal adjunct for use in spinal surgery, the spinal adjunct comprising:
a configured sheet made of a biocompatible mesh material;
the configured sheet formed for attachment onto one or more vertebrae of vertebrae relating to a previous or concurrent spinal procedure; and
the configured sheet sized to be received about an area about the one or more vertebrae of vertebrae relating to a previous or concurrent spinal procedure.

12. The spinal adjunct of claim 1, wherein the biocompatible mesh material comprises PEEK.

13. The spinal adjunct of claim 11, wherein the configured sheet is sized to extend from an upper vertebra to a lower vertebra of the one or more vertebrae of vertebrae relating to a previous or concurrent spinal procedure.

14. The spinal adjunct of claim 13, wherein the configured sheet extends radially about the upper and lower vertebrae of the one or more vertebrae relating to a previous or concurrent spinal procedure.

15. The spinal adjunct of claim 11, wherein the configured sheet is used in conjunction with a spinous process band that is received on and extends between adjacent vertebrae of the one or more vertebrae relating to a previous or concurrent spinal procedure.

16. The spinal adjunct of claim 15, wherein the spinous process band is formed of PEEK.

17. The spinal adjunct of claim 11, wherein the configured sheet is fashioned for attachment onto one or more vertebrae of vertebrae relating to a previous or concurrent spinal procedure by one or more of staples, an adhesive, screws, wiring, and tying.

18. The spinal adjunct of claim 11, wherein the configured sheet is fashioned for introduction to the one or more vertebrae of vertebrae relating to a previous or concurrent spinal procedure through minimal invasive surgery.

19. The spinal adjunct of claim 11, wherein the configured sheet is fashioned for introduction to the one or more vertebrae of vertebrae relating to a previous or concurrent spinal procedure when rolled up.

20. An article for use in spinal surgery, the article comprising:
a flexible mesh sheet formed from PEEK;
wherein the flexible mesh sheet is configured to attachment to one or more vertebrae of vertebrae relating to a previous or concurrent spinal procedure through minimally invasive surgery.
